(19) United States
(12) Patent Application Publication
(10) Pub. No.: US 2017/0280742 A1
(43) Pub. Date: Oct. 5, 2017

(54) METHOD OF FERMENTING COCOA BEANS

(71) Applicant: DUPONT NUTRITION BIOSCIENCES APS, Copenhagen (DK)

(72) Inventors: PASCAL FOURCASSIE, Poitiers (FR); PATRICK BOYAVAL, LA MEZIERE (FR); CLAIRE BONARD, Chatellerault (FR)

(21) Appl. No.: 15/507,505

(22) PCT Filed: Aug. 27, 2015
(86) PCT No.: PCT/EP2015/069660

§ 371 Date: Feb. 28, 2017

(30) Foreign Application Priority Data
Aug. 29, 2014 (EP) 14182818.6

(51) Int. Cl. A23G 1/02; C12N 1/02; C12N 1/16; C12N 1/20; A23G 1/00; A23G 1/56

(52) U.S. Cl. CPC A23G 1/02; A23G 1/003; A23G 1/56; C12N 1/16; C12N 1/20; C12N 1/02; C12N 1/00

(57) ABSTRACT

Use of a yeast capable of producing ethanol and acetic acid by fermentation of a carbohydrate, in a method for the fermentation of cocoa beans, is disclosed.

### FIELD OF THE INVENTION

This invention relates to a method for the fermentation of cocoa beans. It also relates to the use of a yeast in such a method.

### BACKGROUND TO THE INVENTION

Cocoa products are produced from beans of the cocoa tree, Theobroma cacao. Mature fruits (pods) rise directly from the stem of the cocoa tree and are thick walled and contain 30-40 beans (seeds). Each bean consists of two cotyledons and an embryo (radicle) surrounded by a seed coat (testa) and is enveloped in a sweet, white, mucilaginous pulp that comprises approximately 40% of seed fresh weight.

The post-harvesting processing of the cocoa beans starts by opening the pods and removing the seeds from the fruit for fermentation. After fermentation, beans can be washed and then dried. Drying is usually carried out in the sun and takes from 1 to 3 weeks. Once dried to 6-7% humidity, beans are stored in bags in warehouses until being sold and exported.

The fermentation of cocoa has a number of purposes. Firstly, it facilitates removal of the pulp. Secondly, it produces ethanol and acetic acid, which diffuse into the beans: together with the heat produced this causes the death of the seed embryo. Thirdly, it causes bean vegetal cell wall breakdown and liberation of endogenous enzymes induce biochemical reactions within the bean that lead to precursors of chocolate flavour.

Conventionally, cocoa is fermented spontaneously by a consortium of naturally occurring yeasts, lactic acid bacteria (LAB) and acetic acid bacteria (AAB). The actual fermentation takes place in the mucilaginous fruit pulp surrounding the cocoa beans and lasts from 5 to 7 days.

According to R. F. Schwan and A. E. Wheals, Critical Reviews in Food Science and Nutrition, 2004, 44, 205-221, cocoa pulp contains 82-87% water, 10-15% sugars (glucose, fructose and sucrose), 2-3% pentosans, 1-1.5% pectin, 3% citric acid, proteins, amino acids, vitamins, minerals. Its initial pH is around 3.6.

The initial phase of fermentation is dominated by yeasts and lactic acid bacteria. Alcoholic fermentation by yeasts causes the transformation of sugars into ethanol and CO₂. In addition, fermentation involves citric acid degradation, pectin degradation and the production of volatiles (principally fusel alcohols, fatty acids, and fatty acid esters).

Lactic acid fermentation by lactic acid bacteria mainly involves the transformation of sugars into lactic acid (homolactic fermentation), although some species of lactic acid bacteria transform glucose into lactic acid, acetic acid, ethanol and CO₂ (heterolactic fermentation). Fructose can be reduced into mannitol by some lactic acid bacteria, and citric acid assimilation may also take place. Lactic acid fermentation is favoured by low pH, high sugar content, low ethanol and can therefore take place at the end of the alcoholic fermentation. Citric acid conversion results in a rise in pH and a possible contribution to cocoa flavour.

Acetic acid fermentation is carried out by acetic acid bacteria. It involves the oxidation of ethanol into acetic acid. Although the fermentation of sugars into ethanol is anaerobic (i.e. can take place in the absence of oxygen), the conversion of ethanol into acetic acid is aerobic and requires the presence of oxygen.

It is generally understood that both alcoholic and acetic acid fermentations may be necessary to obtain a good quality cocoa. However, lactic acid fermentation is not always required, as when lactic acid enters the cotyledon it is unable to evaporate and can therefore affect the quality of the cocoa bean.

Pulp degrading enzymes cause liquid known as “sweatings” to drain away. R. Buamah et al, World Journal of Microbiology and Biotechnology, 1997, 13, 457-462, describes the effect of a yeast culture on fermentation of cocoa and its effect on the yield of sweatings. In particular, the article describes the use of specific ATCC strains of Kluyveromyces fragilis, Saccharomyces chevalieri, Candida norvengensis and Torulopsis candida. This document describes two experiments: the first using a yeast or a combination of yeasts in aseptic conditions (i.e. no other microorganisms were present); the second a normal spontaneous fermentation without added yeast.

WO 2013/064678 describes the use of a yeast starter of the species Pichia kluyveri in cocoa fermentation. The fermentation is carried out on a laboratory scale. However, it does not disclose that this yeast is able to produce both ethanol and acetic acid by carbohydrate fermentation.

J. Sanchez et al, Lebensmittel Wissenschaft and Technologie, 1985, 18(2), 69-75, describes fermentations carried out with pure yeast cultures of the species Kluyveromyces fragilis, Candida zeylanoides and Saccharomyces chevalieri, the conditions being otherwise the same as those of spontaneous fermentation (which was used as a control). However, the results using Kluyveromyces fragilis showed no improvement compared with spontaneous fermentation and those using Candida zeylanoides were modest. Moreover, this document does not disclose that any of the yeasts are able to produce both ethanol and acetic acid by carbohydrate fermentation.

J. E. Sanchez Vasquez, Café Cacao Thé, 1989, vol. XXXIII, no. 3, 157-163, describes an attempt to improve cocoa fermentation by direct inoculation with micro-organisms. This document describes two experiments: the first using the acetic acid bacterium Acetobacter sp., the other using a yeast of species Brettanomyces claussenii. However, in this second experiment there is considerable contamination of acetic acid bacteria, so there is no disclosure that this yeast is able to produce both ethanol and acetic acid by carbohydrate fermentation. In addition, the results of this experiment show that the population of yeast reaches its maximum on the first day of fermentation, but the yeast has disappeared or died by the fourth day. Moreover, compared with a spontaneous fermentation under the same conditions, the fermentation duration is not reduced.

It is known, in particular from the beverage industry such as beer and wine industries, that certain species of yeasts are able to produce both ethanol and organic acids, including acetic acid, by carbohydrate fermentation. However, these yeasts are unwanted in these industries since they cause spoilage of alcoholic beverages.

Moreover, there is still a need for microorganisms which can improve fermentation of cocoa beans. It would be desirable in cocoa fermentation processes to introduce a microorganism which is capable of catalysing both ethanol and acetic acid production.

### SUMMARY OF THE INVENTION

Therefore, in one aspect, the invention comprises a method for the fermentation of cocoa beans comprising the steps of:
a) adding at least one yeast to a plant material comprising:
   (i) beans derived from fruit pods of the species Theobroma cacao; and
   (ii) pulp; and
b) fermenting the plant material to obtain fermented cocoa beans, the fermentation environment containing at least one other microorganism selected from the group consisting of yeast, lactic acid bacteria and acetic acid bacteria;

wherein the added yeast is able to produce ethanol and acetic acid by fermentation of a carbohydrate.

In another aspect, there is provided according to the invention fermented cocoa beans obtained or obtainable by the above method.

In another aspect, there is provided according to the invention a method of producing a cocoa-based product, comprising:
(a) providing fermented cocoa beans obtained or obtainable by the above method; and
(b) carrying out one or more further processing steps on said fermented cocoa beans to produce the cocoa-based product.

In another aspect, there is provided according to the invention a cocoa-based product obtained or obtainable by the above method. The cocoa-based product may be a food product, such as chocolate, or a non-food product.

In another aspect, there is provided according to the invention use of a yeast capable of producing ethanol and acetic acid by fermentation of a carbohydrate, in a method for the fermentation of cocoa beans, in an environment containing at least one other microorganism selected from the group consisting of yeast, lactic acid bacteria and acetic acid bacteria.

### Advantages

Without wishing to be bound by theory, it is believed that the addition to a cocoa fermentation process of a yeast capable of producing both ethanol and acetic acid according to the present invention will result in production of acetic acid earlier than during conventional spontaneous fermentation without addition of yeast. This has the potential to reduce the time required to carry out the cocoa fermentation process, for example from the current 6 to 7 days using spontaneous fermentation to between 4 and 5 days.

Furthermore, without wishing to be bound by theory, it is believed that the addition to a cocoa fermentation process of a yeast capable of producing both ethanol and acetic acid according to the present invention, will limit the development of other micro-organisms involved in the fermentation process: in particular it is believed that the development of acetic acid bacteria during the process will be limited, compared with conventional spontaneous fermentation without addition of yeast.

In particular, it is believed that the addition of yeast in cocoa fermentation according to the present invention maintains the level of yeast throughout the entire fermentation process. This will increase the count ratio (total yeast count/total acetic acid bacteria count) during the entire fermentation process compared with the same ratio in a spontaneous fermentation environment without addition of yeast. This would run contrary to the teaching of the prior art, which generally describes that even when yeast is added, the yeast count decreases during the fermentation process and the yeast/acetic acid bacteria count ratio reverts to the normal levels observed during spontaneous fermentation.

### BRIEF DESCRIPTION OF THE DRAWINGS

FIG. 1 shows the production of acetic acid (g/L) at 48 hours and at 30° C. by 10 yeasts, according to example 1 (grey) or example 3 (black).

FIG. 2 shows the production of ethanol (g/L) at 48 hours and at 30° C. by 10 yeasts, according to example 1 (grey) or example 3 (black).

FIG. 3 shows the kinetics of acetic acid and ethanol production (g/L) of a Dekerra bruxellensis strain, its consumption of glucose and fructose as well as the OD600 nm according to time (hours).

FIG. 4 shows the kinetics of production of (A) acetic acid (g/L) and (B) ethanol (g/L), as well as (C) the OD at 600 nm of two S. cerevisiae and 1 S. bayanus strains, according to time (hours).

FIG. 5 shows (A) the kinetics of production of acetic acid and ethanol (g/L), as well as (B) the OD at 600 nm of two K. marxianus strains, according to time (hours).

FIG. 6 shows (A) the kinetics of production of acetic acid and ethanol (g/L), as well as (B) the OD at 600 nm of a C. kefyr strain, according to time (hours).

### DETAILED DESCRIPTION

Addition of at Least One Yeast

The method of the invention comprises the addition to a cocoa fermentation environment of a yeast capable of producing both ethanol and acetic acid by carbohydrate fermentation. The fermentation environment comprises cocoa beans (i.e. beans derived from fruit pods of the species Theobroma cacao) and pulp. The nature of the pulp is described below.

Addition of the at least one yeast (step a), under any form, is done in order than said at least one yeast is in contact with a maximum of cocoa beans.

In one embodiment, the yeast is added under a liquid form onto the cocoa beans. In one embodiment, when the yeast is available under a powder form (dried or freeze-dried), yeast culture is previously rehydrated, for example into a sterile saline solution. In one embodiment, when yeast is under a frozen form, yeast culture is previously thawed and optionally diluted before the addition step.

In one embodiment, when the yeast is available under a powder form or a frozen form, the yeast is, prior to be added under a liquid form onto the cocoa beans, propagated on a medium comprising cocoa pulp and fruit juice (such as coco water, banana juice or pineapple juice).

In one embodiment, the yeast is added under a powder form (dried or freeze-dried) onto the cocoa beans.

The addition of said at least one yeast is for example done by pouring or spraying the liquid or the powder onto the cocoa beans.

In one optional embodiment, after the addition of the at least one yeast, cocoa beans are mixed to ensure good homogenization of the added yeast(s).

The at least one yeast is added to the fermentation environment in a count sufficient to enable both ethanol and acetic acid to be produced during the fermentation. Typically, the yeast is added in a count of 10⁴ to 10⁹, preferably 10⁵ to 10⁸ colony forming units (cfu) per gram of plant material (beans+pulp). In one embodiment, the yeast is added in a count of at least 10⁴, 10⁵, 10⁶, 10⁷, 10⁸ cfu/gram of plant material.

In addition to the addition of at least one yeast as described herein at the starting of fermentation, the process may also include the further addition of at least one yeast as described herein, after the fermentation has begun. Therefore, the process comprises also the step of adding during the step (b) of fermentation at least one yeast as described in the invention. In one embodiment, the yeast added during the step (b) of fermentation is the same as the one added during step a). In another embodiment, the yeast added during the step (b) of fermentation is different from the one added during step a), but is still as described herein. The skilled person can determine whether there is a need for addition of at least one strain during step (b) of fermentation, for example by determining that the level of ethanol produced is insufficient, and/or that the level of acetic acid produced is insufficient. The at least one yeast may be added at any time during step (b) of fermentation, and preferably during the first 24 hours, the first 48 hours or the first 96 hours of fermentation. In a particular embodiment, the at least one yeast is added 24 hours, 48 hours or 96 hours after step a), in particular between 18 hours and 30 hours, more particularly between 22 hours and 26 hours after step a).

Fermentation Process

Fermentation typically causes the formation of chocolate precursor compounds. These react with one another during subsequent processing (particularly thermal treatment such as roasting of the beans) as described below, to form the compounds responsible for the flavour of chocolate.

At the beginning of the process of the invention, both beans and pulp are present. The process begins with the addition of yeast to the fermentation process. In one embodiment, the process ends when at least part of the pulp is consumed (i.e. at least a portion of the fermentable carbohydrates in the pulp have been converted to ethanol and acetic acid). In one embodiment, the process ends when at least part of the pulp is consumed (i.e. all of the fermentable carbohydrates in the pulp have been converted to ethanol and acetic acid). In one embodiment, the process ends when the pulp is completely liquefied. In one embodiment, the process ends when almost all the pulp (at least 80%, at least 90% or at least 95%) is liquefied.

In one embodiment the fermentation is carried out in heaps, wooden boxes, trays or baskets to allow for spontaneous fermentation to occur.

The temperature at which the process of the invention may be carried out typically varies from 20° C. to 60° C., and preferably 30° C. to 50° C. In one embodiment, the temperature at which the process of the invention may be carried out is above 30° C., preferably above 35° C.

In particular, without wishing to be bound by theory, it is believed that, in contrast to the processes of the prior art, the added yeast used in the present invention can catalyse the production of both ethanol and acetic acid concomitantly. This can accelerate the fermentation process. Typically, the fermentation step (b) takes from 2 to 7 days, more preferably 4 or 5 days. Using the method of the present invention, the fermentation takes at least 1 day, preferably at least 2 days less than a corresponding fermentation process but without yeast addition.

In one embodiment the beans are turned during the fermentation process. This ensures an adequate air supply around the fermenting beans. In one embodiment the turning process is carried out once per day of the fermentation process.

The fermentation environment typically contains a wide range of microorganisms. The microorganisms present comprise at least one of yeasts, lactic acid bacteria and/or acetic acid bacteria. Typically, the microorganisms in the fermentation environment comprise yeasts and acetic acid bacteria. In one embodiment the microorganisms in the fermentation environment comprise yeast, lactic acid bacteria and acetic acid bacteria.

In one embodiment, the fermentation environment is a spontaneous fermentation environment. In this specification the term “spontaneous fermentation environment” in its broadest sense means the environment typically found in the conventional cocoa fermentation processes (i.e., without the addition of any microorganism). An example of spontaneous fermentation is disclosed in “Qualité du cacao, L'impact du traitement post-récolte”; edition Quæ, 2013).

In one embodiment, the method of the invention is carried out outdoor, i.e., in the natural conditions in the places where the cocoa beans are collected. In one embodiment, the method of the invention is not carried out under aseptic conditions. In one embodiment the spontaneous fermentation environment is in the same conditions as on-farm processing, but without addition of yeast. In one embodiment the spontaneous fermentation environment contains the same cocoa beans as the conventional cocoa fermentation processes, but without addition of yeast. In one embodiment the spontaneous fermentation environment contains the same pulp as the conventional cocoa fermentation processes, but without addition of yeast. In one embodiment the spontaneous fermentation environment is at the same temperature as the conventional cocoa fermentation processes, but without addition of yeast. In one embodiment the spontaneous fermentation environment contains the same microorganisms typically as the conventional cocoa fermentation processes (including but not limited to yeasts, acetic acid bacteria, and lactic acid bacteria), but without addition of yeast. In one embodiment the spontaneous fermentation environment contains the same indigenous flora as the conventional cocoa fermentation processes (including but not limited to yeasts, acetic acid bacteria, and lactic acid bacteria), but without addition of yeast. In one embodiment the spontaneous fermentation environment uses the same covering plant material as the conventional cocoa fermentation processes of the prior art, but without addition of yeast.

In one embodiment, the acetic acid is produced by acetic acid bacteria in the fermentation environment (with added yeast) in an amount less than the amount of acetic acid produced in a spontaneous fermentation environment without addition of yeast.

The added yeast typically produces the majority of the acetic acid produced during the fermentation step, the other part of the acetic acid being produced by the indigenous acetic acid bacteria. In one embodiment the added yeast produces more than 50% of the acetic acid produced during the fermentation step. In one embodiment the added yeast produces more than 60% of the acetic acid produced during the fermentation step. In one embodiment the added yeast produces more than 70% of the acetic acid produced during the fermentation step. In one embodiment the added yeast produces more than 80% of the acetic acid produced during the fermentation step. In one embodiment, the added yeast, when producing more than 50%, 60%, 70% or 80% of the acetic acid produced during the fermentation step, produces up to 90% of the acetic acid produced during the fermentation step. In a particular embodiment, the added yeast produces between 50 and 80%, 50 and 90%, 60 and 80%, 60 and 90%, 70 and 80%, 70 and 90% or 80 and 90% of the acetic acid produced during the fermentation step. In a particular embodiment, the indigenous acetic acid bacteria produce less than 10% or less than 20% of the acetic acid produced during the fermentation step, the majority of the acetic acid produced during the fermentation step being produced by the added yeast.

In one embodiment, the ratio of (total yeast count/total acetic acid bacteria count) during the entire process of the invention is higher than the corresponding ratio in a spontaneous fermentation environment without addition of yeast. Without wishing to be bound by theory, it is believed that, in contrast to the prior art, where the yeast count generally decreases as the fermentation process proceeds, the maintenance of high yeast levels, in particular a high ratio of yeast/acetic acid bacteria throughout the process enables the levels of acetic acid bacteria to be controlled and the duration of the fermentation process shortened.

In one embodiment the ratio total yeast/total acetic acid bacteria (log/log) (referred to herein as the “Y/AAB ratio”, the ratio being calculated based on the logarithm to base 10 of the number of colony forming units of each of the microorganisms) present in the fermentation environment during step (b) equals to or is more than 0.8. In one embodiment, the Y/AAB ratio equals to or is more than 0.8 during the 3 first days of fermentation. In one embodiment, the Y/AAB ratio equals to or is more than 0.8 during the 4 first days of fermentation. In one embodiment, the Y/AAB ratio equals to or is more than 0.8 during the 5 first days of fermentation. In one embodiment, the Y/AAB ratio equals to or is more than 0.9. In one embodiment, the Y/AAB ratio equals to or is more than 1. Without wishing to be bound by theory, it is believed that, in contrast to the prior art, where the yeast count generally decreases as the fermentation process proceeds, the maintenance of high yeast populations enables yeast to grow in preference to acetic acid bacteria and the duration of the fermentation process consequently shortened.

Beans

The process of the invention is carried out on cocoa beans, i.e. beans derived from the fruit pods of the tree Theobroma cacao. During the process, the ethanol and acetic acid produced during the fermentation penetrate the bean so as to kill the embryo in the bean and generate chocolate flavour precursors.

Typically, the process is carried out on heaps, boxes (such as wooden boxes), trays or baskets typically weighing from 20 kg to 2000 kg. Typically, the beans are enclosed by plant material such as banana or plantain leaves, the function of which to conserve the heat generated during the fermentation process. Typically, the dimensions of such boxes are designed so as to allow for optimal heat insulation and air transfers.

Pulp

The fermentation process of the invention involves the addition of yeasts to pulp. The pulp both acts as a nutrient source for the added yeast and other microorganisms, and as a source of carbohydrates for the production of the ethanol and acetic acid formed in the fermentation process. These and other compounds (such as aromatic molecules) penetrate the beans and cause the formation of cocoa flavour and cocoa flavour precursors.

The nature of the pulp is not particularly limited provided it is contains one or more fermentable carbohydrates which the fermentation micro-organisms are capable of acting upon to produce ethanol and acetic acid. Typical carbohydrates included in the pulp include monosaccharides such as glucose and fructose, disaccharides such as sucrose, and polysaccharides such as pectin, cellulose, hemicellulose and lignin.

In one embodiment, the pulp comprises cocoa pulp, i.e. the pulp naturally surrounding the cocoa bean. In one embodiment, part of the cocoa pulp (at most 10%, at most 20% or between 10 and 20%) is removed during or prior to the addition of the yeast. Without wishing to be bound by theory, it is believed that, by limiting the initial pulp content, it may be possible to control the level of acetic acid produced using the process of the invention.

In one embodiment, the pulp comprises a mixture of cocoa pulp together with a pulp from a plant other than Theobroma cacao. In one embodiment, the pulp consists essentially of, or consists of, only the pulp from a plant other than Theobroma cacao, i.e. the cocoa pulp is completely removed. The nature of the other plant is not particularly limited provided its pulp contains one or more fermentable carbohydrates which the fermentation micro-organisms are capable of acting upon to produce ethanol and acetic acid.

Yeast

Yeast taxonomy used herein is according to “The yeasts: a taxonomic study” 5th edition; Elsevier, 2011.

According to the present invention, a yeast is added to a cocoa fermentation process (such as a natural fermentation process). In one embodiment, the added yeast is a single strain of yeast. In one embodiment, the yeast comprises a mixture of yeasts.

In one embodiment, the added yeast is of the same species as the yeast naturally present in a spontaneous fermentation environment. However, it is generally preferred according to the present invention that the yeast is different from the yeasts naturally present in a spontaneous fermentation environment, as yeasts involved in natural cocoa fermentation are generally unable to produce both ethanol and acetic acid. In one embodiment, it is generally preferred according to the present invention that the added yeast is different from the yeasts naturally present in the spontaneous fermentation environment where said yeast is added. Thus, in one embodiment the yeast which is added in a particular spontaneous fermentation environment (where this species is naturally not present) comes from another particular spontaneous fermentation environment. Indeed, it is known that some yeast species are present only in the fermentation environment of some countries or geographical areas (Table 2 of “Qualité du cacao, L'impact du traitement post-récolte”; edition Quæ, 2013). As a particular example, an added yeast, coming from an African spontaneous fermentation environment, is used within a fermentation method of the invention in an Asian spontaneous fermentation environment, or vice versa. As another particular example, an added yeast, coming from the spontaneous fermentation environment of a first country, is used within a fermentation method of the invention in the spontaneous fermentation environment of a second country.

The added yeasts used in the present invention are those yeasts which are capable of producing both ethanol and acetic acid by carbohydrate fermentation.

The expression “yeast which are capable of producing both ethanol and acetic acid” encompasses both yeasts which are capable of producing (or produce) low level of ethanol and high level of acetic acid, and yeasts which are capable of producing (or produce) both high level of ethanol and high level of acetic acid. In the present invention, yeasts which are capable of producing (or produce) both high level of ethanol and high level of acetic acid are preferred.

Within the present invention, a yeast is considered to produce high level of acetic acid when:
the concentration of acetic acid produced by said yeast during assay I equals to or is above 0.7, 0.8, 0.9, 1, 1.1, 1.2, 1.3, 1.4, 1.5, 2 or 2.5 g/L, and/or
the ratio of the concentration of acetic acid produced by said yeast during assay I over the final OD (i.e., the OD600ₙₘ at the end of assay I) equals to or is above 50, 60, 70, 80, 90, 100, 120, 150, 200, 250 or 300 mg/L/OD.

Within the present invention, a yeast is considered to produce high level of ethanol, when:
the concentration of ethanol produced by said yeast during assay I equals to or is above 30, 35, 40, 45 or 50 g/L, and/or
the ratio of the concentration of ethanol produced by said yeast during assay I over the final OD (i.e., the OD600ₙₘ at the end of assay I) equals to or is above 3, 3.5 or 4 g/L/OD.

In one embodiment, a yeast used in the present invention is able to produce acetic acid during assay I in a concentration equal to or above 0.7, 0.8, 0.9, 1, 1.1, 1.2, 1.3, 1.4, 1.5, 2 or 2.5 g/L, preferably equal to or above 1, 1.1, 1.2, 1.3, 1.4, 1.5, 2 or 2.5 g/L, more preferably equal to or above 1.5, 2 or 2.5 g/L.

In one embodiment, alone or in combination with the previous one, a yeast used in the present invention is able to produce acetic acid during assay I in a ratio of the concentration of acetic acid produced by said yeast over the final OD equal to or above 50, 60, 70, 80, 90, 100, 120, 150, 200, 250 or 300 mg/L/OD, preferably equal to or above 100, 120, 150, 200, 250 or 300 mg/L/OD, more preferably equal to or above 150, 200, 250 or 300 mg/L/OD.

In one embodiment, and in addition to produce acetic acid at a high level as described in the two previous paragraphs, a yeast used in the present invention is able to produce ethanol during assay I in a concentration equal to or above 30, 35, 40, 45 or 50 g/L, preferably equal to or above 40, 45 or 50 g/L, more preferably equal to or above 45 or 50 g/L.

In one embodiment, alone or in combination with the previous one, a yeast used in the present invention is able to produce ethanol during assay I in a ratio of the concentration of ethanol produced by said yeast over the final OD equal to or above 3, 3.5 or 4 g/L/OD.

In one embodiment, a yeast used in the present invention is able:

(a) to produce acetic acid during assay I in a concentration equal to or above 1, 1.1, 1.2, 1.3, 1.4, 1.5, 2 or 2.5 g/L, preferably equal to or above 1.5, 2 or 2.5 g/L, and/or to produce acetic acid during assay I in a ratio of the concentration of acetic acid produced by said yeast over the final OD equal to or above 100, 120, 150, 200, 250 or 300 mg/L/OD, preferably equal to or above 150, 200, 250 or 300 mg/L/OD, and

(b) optionally, to produce ethanol during assay I in a concentration equal to or above 40, 45 or 50 g/L, more preferably equal to or above 45 or 50 g/L and/or to produce ethanol during assay I in a ratio of the concentration of ethanol produced by said yeast over the final OD equal to or above 3, 3.5 or 4 g/L/OD.

In one embodiment, a yeast used in the present invention is able:

(a) to produce acetic acid during assay I in a concentration equal to or above 1.5, 2 or 2.5 g/L, and/or to produce acetic acid during assay I in a ratio of the concentration of acetic acid produced by said yeast over the final OD equal to or above 150, 200, 250 or 300 mg/L/OD, and

(b) optionally, to produce ethanol during assay I in a concentration equal to or above 45 or 50 g/L, and/or to produce ethanol during assay I in a ratio of the concentration of ethanol produced by said yeast over the final OD equal to or above 3, 3.5 or 4 g/L/OD.

In one embodiment, a yeast used in the present invention is able (a) to produce acetic acid during assay I in a concentration equal to or above 1, 1.1, 1.2, 1.3, 1.4, 1.5, 2 or 2.5 g/L, preferably equal to or above 1.5, 2 or 2.5 g/L, and optionally, to produce ethanol during assay I in a concentration equal to or above 40, 45 or 50 g/L, more preferably equal to or above 45 or 50 g/L. In one embodiment, a yeast used in the present invention is able (a) to produce acetic acid during assay I in a concentration equal to or above 1.5, 2 or 2.5 g/L, and optionally, to produce ethanol during assay I in a concentration equal to or above 45 or 50 g/L. In one embodiment, a yeast used in the present invention is able (a) to produce acetic acid during assay I in a concentration equal to or above 1, 1.1, 1.2, 1.3, 1.4, 1.5, 2 or 2.5 g/L, preferably equal to or above 1.5, 2 or 2.5 g/L, and to produce ethanol during assay I in a concentration equal to or above 40, 45 or 50 g/L, more preferably equal to or above 45 or 50 g/L. In one embodiment, a yeast used in the present invention is able (a) to produce acetic acid during assay I in a concentration equal to or above 1.5, 2 or 2.5 g/L, and to produce ethanol during assay I in a concentration equal to or above 45 or 50 g/L.

Values for the level of acetic acid (g/L), the level of ethanol (g/L) and ratios (mg/L/OD and g/L/OD) are expressed herein with a relative standard deviation of ±10%.

From the species capable of producing both ethanol and acetic acid by carbohydrate fermentation, examples of suitable yeasts are described herein, and include those selected according to the assay referred to as Example 1 herein (assay I).

In one embodiment, the added yeast is of a genus selected from the group consisting of Dekkera, Brettanomyces, Hanseniaspora, Kloeckera, Pichia, Candida, Saccharomyces, Kluyveromyces, Debaryomyces, Kazachstania, Wickerhamomyces, Lindnera and Zygotorulaspora, or any mixture thereof.

In one embodiment, the added yeast is of the genus Dekkera.

In one embodiment, the added yeast is of the genus Brettanomyces. In one embodiment, when the added yeast is of genus Brettanomyces, said yeast is not of the species Brettanomyces claussenii (today named Brettanomyces anomalus), or when in a combination of more than one yeast does not comprise the species Brettanomyces claussenii (today named Brettanomyces anomalus).

In one embodiment, the added yeast is of the genus Hanseniaspora.

In one embodiment, the added yeast is of the genus Kloeckera.

In one embodiment, the added yeast is of the genus Pichia. In one embodiment, when the added yeast is of the genus Pichia, the added yeast is not of the species Pichia kluyveri. In one embodiment, the added yeast is of the genus Candida. In one embodiment, when the added yeast is of the genus Candida, the added yeast is not of the species Candida incommunis (also known as C. norvengenis) and/or Candida zeylanoides and/or is not the strain ATCC20031.

In one embodiment, the added yeast is of the genus Saccharomyces. In one embodiment, when the added yeast is of genus Saccharomyces, the added yeast is not of the species S. cerevisiae.

In one embodiment, the added yeast is of the genus Kluyveromyces. In one embodiment, when the added yeast is of genus Kluyveromyces, the added yeast is not of the species Kluyveromyces marxianus.

In one embodiment, the added yeast is of the genus Debaryomyces.

In one embodiment, the added yeast is of the genus Kazachstania.

In one embodiment, the added yeast is of the genus Wickerhamomyces.

In one embodiment, the added yeast is of the genus Lindnera.

In one embodiment, the added yeast is of the genus Zygotorulaspora.

In one embodiment, the added yeast is selected among one or several of the following species:
Dekkera bruxellensis (anamorph Brettanomyces bruxellensis),
Brettanomyces custersianus,
Brettanomyces nanus,
Brettanomyces naardenensis,
Hanseniaspora uvarum (anamorph Kloeckera apiculata),
Hanseniaspora guillermondii (anamorph Kloeckera apis),
Hanseniaspora osmophila (anamorph Kloeckera corticis),
Pichia fermentans (anamorph Candida lambica),
Pichia membranifaciens (anamorph Candida valida),
Wickerhamomyces anomalus (previously known as Pichia anomala) (anamorph Candida pelliculosa),
Pichia kluyveri; in one embodiment, when the yeast is of the species Pichia kluyveri, said yeast is not the strain Pichia kluyveri PK-KR1 and/or the strain Pichia kluyveri PKKR2, deposited on 24 Aug. 2006 at the National Measurement Institute, under accession numbers V06/022711 and V06/022712,
Kluyveromyces lactis (anamorph Candida spherica),
Debaryomyces hansenii (anamorph Candida famata),
Lindnera jadinii (anamorph Candida utilis),
Kazachstania unispora (also known as Saccharomyces unisporus),
Zygotorulaspora florentina (previously known as Saccharomyces florentinus),
Saccharomyces bayanus.

In one embodiment, the yeast is of the species Kluyveromyces marxianus (Candida kefyr) (also known as K. fragilis); in one embodiment, when the yeast is from the species K. marxianus, said yeast is not the Kluyveromyces fragilis strain available at the ATCC under number 8601 and/or the K. marxianus strain deposited under CBS1555 (Centraal Bureau voor Schimmelcultures).

In one embodiment, the yeast is of the species Candida zeylanoides.

In one embodiment, the yeast is of the species Candida incommunis. In one embodiment, when the yeast is of the species Candida incommunis, the yeast is not the Candida incommunis strain deposited under number CBS5604 (or ATCC22971).

In one embodiment, the yeast is of the species Saccharomyces cerevisiae (also known as Saccharomyces chevaliers). In one embodiment, when the yeast is from the species S. cerevisiae, the yeast is not the S. cerevisiae strain deposited at the ATCC under number 52712.

In one embodiment, the yeast is of the species Brettanomyces anomalus (also known as Dekerra anomala or Brettanomyces claussenii). In one embodiment, when the yeast is from the species Brettanomyces anomalus, the yeast is not the Brettanomyces anomalus strain referred to in J. Sanchez et al, Lebensmittel Wissenschaft and Technologie, 1985, 18(2), 69-75, as C-Y-31-2-2.

In one embodiment, yeast used within the present invention is selected from the group consisting of Saccharomyces bayanus, Saccharomyces cerevisiae, Kluyveromyces marxianus (Candida kefyr), Dekkera bruxellensis, Hanseniaspora guillermondii, Pichia kluyveri, Pichia anomala and any mixture thereof, provided that when:
this yeast is Saccharomyces cerevisiae and is used alone, this yeast is not S. cerevisiae strain deposited at the ATCC under number 52712,
this yeast is Kluyveromyces marxianus and is used alone, this yeast is not the Kluyveromyces fragilis strain available at the ATCC under number 8601 and/or the K. marxianus strain deposited under CBS1555, and
this yeast is Pichia kluyveri and is used alone, this yeast is not Pichia kluyveri PK-KR1 and/or the strain Pichia kluyveri PKKR2, deposited on 24 Aug. 2006 at the National Measurement Institute, under accession numbers V06/022711 and V06/022712.

In one embodiment, the at least one added yeast used within the present invention is selected from the group consisting of Saccharomyces bayanus, Dekkera bruxellensis, Pichia anomala and any mixture thereof. In one embodiment, the at least one added yeast used within the present invention is selected from the group consisting of Saccharomyces bayanus, Dekkera bruxellensis and any mixture thereof. In one embodiment, the at least one added yeast used within the present invention is selected from the group consisting of Saccharomyces bayanus and any mixture of yeasts comprising S. bayanus. In one embodiment, the at least one added yeast used within the present invention is selected from the group consisting of Dekkera bruxellensis and any mixture of yeasts comprising D. bruxellensis.

In one embodiment, the yeast preferably used within the present invention is a yeast which produce high level of acetic acid and high level of ethanol (as defined herein, for example using assay I) and is selected from the group consisting of Saccharomyces bayanus, Saccharomyces cerevisiae, Kluyveromyces marxianus (Candida kefyr) and any mixture thereof, provided that when:
this yeast is Saccharomyces cerevisiae and is used alone, this yeast is not S. cerevisiae strain deposited at the ATCC under number 52712, and
this yeast is Kluyveromyces marxianus and is used alone, this yeast is not the Kluyveromyces fragilis strain available at the ATCC under number 8601 and/or the K. marxianus strain deposited under CBS1555. In one embodiment, the yeast is a Saccharomyces bayanus yeast which produces high level of acetic acid and high level of ethanol (as defined herein).

In one embodiment, the yeast is a yeast which produces high level of acetic acid and high level of ethanol (as defined herein) selected from the group consisting of Saccharomyces bayanus and any mixture of yeasts comprising S. bayanus.

In one embodiment, the yeast preferably used within the present invention is a yeast which produce high level of acetic acid and low level of ethanol (as defined herein, for example using assay I) and is selected from the group consisting of Dekkera bruxellensis, Hanseniaspora guillermondii, Pichia kluyveri, Pichia anomala and any mixture thereof, provided that when this yeast is Pichia kluyveri and is used alone, this yeast is not Pichia kluyveri PK-KR1 and/or the strain Pichia kluyveri PKKR2, deposited on 24 Aug. 2006 at the National Measurement Institute, under accession numbers V06/022711 and V06/022712. In one embodiment, the yeast preferably used within the present invention is a yeast which produce high level of acetic acid and low level of ethanol (as defined herein, for example using assay I) and is selected from the group consisting of Dekkera bruxellensis, Pichia kluyveri, Pichia anomala and any mixture thereof, provided that when this yeast is Pichia kluyveri and is used alone, this yeast is not Pichia kluyveri PK-KR1 and/or the strain Pichia kluyveri PKKR2, deposited on 24 Aug. 2006 at the National Measurement Institute, under accession numbers V06/022711 and V06/022712. In one embodiment, the yeast is a yeast which produces high level of acetic acid and low level of ethanol (as defined herein) and selected from the group consisting of Dekkera bruxellensis, Pichia anomala and any mixture thereof.

In one embodiment, the yeast is a yeast which produces high level of acetic acid and low level of ethanol (as defined herein) selected from the group consisting of Dekkera bruxellensis and any mixture of yeasts comprising D. bruxellensis.

A “mixture of yeast” is defined herein as a blend of at least 2 yeasts, in particular 2, 3 or 4 yeasts. In a particular embodiment, the at least 2 yeasts, in particular the 2, 3 or 4 yeasts, are of different species.

In one embodiment, a mixture of yeasts is added to a cocoa fermentation process, wherein at least one yeast is selected from the yeasts described herein. In one embodiment, the mixture of yeasts comprises or consists of at least 2 yeasts selected from the yeasts described herein. In one embodiment, the mixture of yeasts consists of or comprises at least 2 yeasts, such as 2, 3 or 4 yeasts, each selected independently, from the group consisting of Saccharomyces bayanus, Saccharomyces cerevisiae, Kluyveromyces marxianus (Candida kefyr), Dekkera bruxellensis, Hanseniaspora guillermondii, Pichia kluyveri and Pichia anomala.

In one embodiment, the mixture of yeasts—added to a cocoa fermentation process of the invention—consists of or comprises at least 2 yeasts of different species, such as 2, 3 or 4 yeasts of different species, each selected independently, from the group consisting of Saccharomyces bayanus, Saccharomyces cerevisiae, Kluyveromyces marxianus (Candida kefyr), Dekkera bruxellensis, Hanseniaspora guillermondii, Pichia kluyveri and Pichia anomala.

In a particular embodiment, the mixture of yeasts consists of or comprises at least 2 yeasts of different species, such as 2, 3 or 4 yeasts of different species, wherein one yeast is selected from the group consisting of Saccharomyces bayanus and Dekkera bruxellensis, and one yeast is selected from the group consisting of Saccharomyces bayanus, Dekkera bruxellensis, Kluyveromyces marxianus (Candida kefyr), Saccharomyces cerevisiae, Hanseniaspora guillermondii, Pichia kluyveri and Pichia anomala.

In a particular embodiment, the mixture—added to a cocoa fermentation process of the invention—comprises or consists of at least two yeasts, in particular 2 yeasts, wherein one yeast is a Dekkera bruxellensis yeast and one yeast is selected in the group consisting of Saccharomyces bayanus, Saccharomyces cerevisiae and Kluyveromyces marxianus (Candida kefyr). In these embodiments, the Dekkera bruxellensis preferably produces high level of acetic acid and low level of ethanol (as defined herein).

In one embodiment, the mixture—added to a cocoa fermentation process of the invention—comprises or consists of at least two yeasts, in particular 2 yeasts, wherein one yeast is from the species Saccharomyces bayanus and one yeast is from the genus Pichia, Kluyveromyces, Hanseniaspora or Dekkera or is a yeast from the Saccharomyces genus different from the bayanus species. In one embodiment, the mixture comprises or consists of at least two yeasts, in particular 2 yeasts, wherein one yeast is from the species Saccharomyces bayanus and one yeast is selected from the group consisting of Saccharomyces cerevisiae, Kluyveromyces marxianus (Candida kefyr), Dekkera bruxellensis, Hanseniaspora guillermondii, Pichia kluyveri and Pichia anomala. In one embodiment, the mixture comprises or consists of at least two yeasts, in particular 2 yeasts, wherein one yeast is from the species Saccharomyces bayanus and one yeast is selected from the group consisting of Saccharomyces cerevisiae, Kluyveromyces marxianus (Candida kefyr) and Dekkera bruxellensis. In these embodiments, the Saccharomyces bayanus strain preferably produces high level of acetic acid and high level of ethanol (as defined herein).

In one embodiment, either when used alone or as a mixture, the yeast(s) as defined herein, are suitable to be used in a process of the invention at a temperature above 30° C., preferably above 35° C. Thus, the yeast(s) as defined herein are able to produce acetic acid, and optionally ethanol, in a process of the invention which is carried out at a temperature above 30° C., preferably above 35° C.

In one embodiment, the yeast or mixture of yeasts is the sole microorganism added during the fermentation process. In one embodiment, the yeast or mixture of yeasts is the sole microorganism involved in fermentation of the beans added during the fermentation process.

Other Ingredients

In one embodiment, at least one yeast having a pectinolytic activity is present during at least part of the process. In one embodiment, the at least one yeast having a pectinolytic activity is the added yeast capable of producing both ethanol and acetic acid by carbohydrate fermentation. In another embodiment the yeast having a pectinolytic activity is different from, but added in a common composition with, the added yeast capable of producing both ethanol and acetic acid by carbohydrate fermentation. In another embodiment the yeast having a pectinolytic activity is different from and added independently from the added yeast capable of producing both ethanol and acetic acid by carbohydrate fermentation. An example of yeast having a pectinolytic activity is yeast producing polygalacturonases (PG), pectinmethylesterases (PME) and/or pectin and pectate lyases (PL).

In one embodiment, at least one enzyme having a pectinolytic activity, such as a commercial solution of pectinase, is present during at least part of the process. In one embodiment, the at least one enzyme having a pectinolytic activity is produced in situ by the added yeast capable of producing both ethanol and acetic acid by carbohydrate fermentation. In another embodiment the enzyme having a pectinolytic activity is added in a common composition with the added yeast capable of producing both ethanol and acetic acid by carbohydrate fermentation. In another embodiment at least one enzyme having a pectinolytic activity is added independently from the added yeast capable of producing both ethanol and acetic acid by carbohydrate fermentation.

In one embodiment, no acetic acid bacterium is added during the process of the invention.

In one embodiment, a lactic acid bacterium is added during the process of the invention. The lactic acid bacterium may be any of those normally present during cocoa fermentation, as described and exemplified above. In one embodiment, no lactic acid bacterium is added during the process of the invention. In another embodiment the lactic acid bacterium is added in a common composition with the added yeast capable of producing both ethanol and acetic acid by carbohydrate fermentation. In another embodiment the lactic acid bacterium is added independently from the added yeast capable of producing both ethanol and acetic acid by carbohydrate fermentation.

In one embodiment, further nutrients are added to aid the growth of the yeast and the fermentation process. Examples of such further nutrients include nitrogen and iron.

Fermented Cocoa Beans

The invention also comprises fermented cocoa beans obtained or obtainable by the method of the invention. The fermentation process (particularly the elevated temperature and acid influx) disrupts the cellular structure of the bean and causes compounds present in the bean to mix and react. In particular, reactions between storage proteins, enzymes (such as proteases, polyphenol oxidase and intervase) and result in the formation of chocolate flavour precursors. Examples of chocolate flavour precursors include polypeptides and amino acids (produced by the degradation of proteins), reducing sugars (glucose and fructose) produced by the degradation of sucrose. In addition tannin molecules are formed by the degradation and oxidation of polyphenols, which reduces astringency. Theobromine and caffeine are also diffused and exude from the bean, which also reduces astringency and bitterness.

Subsequent Processing Steps and Cocoa-Based Products

The invention also comprises cocoa-based products obtained or obtainable by the method of the invention, followed by subsequent processing steps as necessary to produce the cocoa-based product. Examples of cocoa-based products include cocoa-based food products, such as chocolate and cocoa beverages (hot chocolate).

The fermented cocoa beans produced according to the invention may undergo a number of further processing steps to make them suitable for chocolate production.

In one embodiment, the further processing step comprises drying the fermented cocoa beans. Flavour forming reactions typically continue throughout the drying step. In addition, strong browning reactions such as oxidation of polyphenols continue to reduce astringency.

In one embodiment, the drying step is a sun drying step or an artificial drying step. Sun drying is preferred as it results in a significant lowering of the acidity: volatile acetic acid escapes through the husk, and non-volatile lactic acid is partly transported by the water from the bean to the husk.

In one embodiment, the further processing step comprises thermally treating the dried beans. Typically the thermal treatment comprises dry roasting which is a process by which heat is applied to the dried beans in the absence of water or air as a carrier. Typically, the dried beans are stirred during dry roasting to ensure even heating.

In one embodiment, the further processing step comprises removing shells (husk) from the roasted beans to produce cocoa nibs. In one embodiment, the further processing step comprises grinding and liquefying the nibs to produce a cocoa liquor.

In one embodiment, the further processing step comprises mixing the cocoa liquor with cocoa butter and sugar to produce chocolate. Further ingredients well known in the art of chocolate production may be added, such as milk or milk solids (to produce milk chocolate), soy lecithin (an emulsifier), and flavourings, such as vanilla, mint or fruit flavourings.

Mixtures of Yeasts

The invention also concerns mixtures of yeasts (as defined herein), and their uses in cocoa bean fermentation.

In one embodiment, the mixture of yeasts consists of or comprises at least 2 yeasts, such as 2, 3 or 4 yeasts, each selected independently, from the group consisting of Saccharomyces bayanus, Saccharomyces cerevisiae, Kluyveromyces marxianus (Candida kefyr), Dekkera bruxellensis, Hanseniaspora guillermondii, Pichia kluyveri and Pichia anomala.

In one embodiment, the mixture of yeasts consists of or comprises at least 2 yeasts of different species, such as 2, 3 or 4 yeasts of different species, each selected independently, from the group consisting of Saccharomyces bayanus, Saccharomyces cerevisiae, Kluyveromyces marxianus (Candida kefyr), Dekkera bruxellensis, Hanseniaspora guillermondii, Pichia kluyveri and Pichia anomala.

In one embodiment, the mixture of yeasts consists of or comprises at least 2 yeasts of different species, such as 2, 3 or 4 yeasts of different species, wherein one yeast is selected from the group consisting of Saccharomyces bayanus and Dekkera bruxellensis, and one yeast is selected from the group consisting of Saccharomyces bayanus, Dekkera bruxellensis, Kluyveromyces marxianus (Candida kefyr), Saccharomyces cerevisiae, Hanseniaspora guillermondii, Pichia kluyveri and Pichia anomala.

In a particular embodiment, the mixture comprises or consists of at least two yeasts, in particular 2 yeasts, wherein one yeast is a Dekkera bruxellensis yeast and one yeast is selected in the group consisting of Saccharomyces bayanus, Saccharomyces cerevisiae and Kluyveromyces marxianus (Candida kefyr). In these embodiments, the Dekkera bruxellensis preferably produces high level of acetic acid and low level of ethanol (as defined herein).

In one embodiment, the mixture comprises or consists of at least two yeasts, in particular 2 yeasts, wherein one yeast is from the species Saccharomyces bayanus and one yeast is from the genus Pichia, Kluyveromyces, Hanseniaspora or Dekkera or is a yeast from the Saccharomyces genus different from the bayanus species. In one embodiment, the mixture comprises or consists of at least two yeasts, in particular 2 yeasts, wherein one yeast is from the species Saccharomyces bayanus and one yeast is selected from the group consisting of Saccharomyces cerevisiae, Kluyveromyces marxianus (Candida kefyr), Dekkera bruxellensis, Hanseniaspora guillermondii, Pichia kluyveri and Pichia anomala. In one embodiment, the mixture comprises or consists of at least two yeasts, in particular 2 yeasts, wherein one yeast is from the species Saccharomyces bayanus and one yeast is selected from the group consisting of Saccharomyces cerevisiae, Kluyveromyces marxianus (Candida kefyr) and Dekkera bruxellensis. In these embodiments, the Saccharomyces bayanus strain preferably produces high level of acetic acid and high level of ethanol (as defined herein).

In one embodiment, the yeast(s) of said mixture are able to produce acetic acid, and optionally ethanol, in a process of the invention which is carried out at a temperature above 30° C., preferably above 35° C.

In one embodiment, the mixture of yeasts of the invention does not comprise acetic acid bacteria and/or lactic acid bacteria, preferably does not comprise acetic acid bacteria.

In one embodiment, optionally in combination with the paragraph immediately above the mixture of yeasts of the invention is in a frozen, dried, freeze-dried, liquid or solid format, in the form of pellets or frozen pellets, or in a powder or dried powder. In one embodiment, the mixture of yeasts of the invention is in a frozen format or in the form of pellets or frozen pellets. In one embodiment, the mixture of yeasts of the invention is in a dried or freeze-dried format, or in a powder or dried powder.

In one embodiment, optionally in combination with the format features above, the mixture of yeasts of the present invention is highly concentrated, such that it can be directly added to or put into contact with the cocoa beans, i.e., possibly without any previous propagation. In an embodiment, and whatever the format, in particular under frozen or dried format, the concentration of yeast(s) is in the range of 10⁵ to 10¹¹ CFU (colony forming units) per g of the mixture, and more preferably at least 10⁶, at least 10⁷, at least 10⁸, at least 10⁹, at least 10¹⁰ or at least 10¹¹ CFU/g of the mixture.

### Examples

### Example 1: Production of Ethanol and Acetic Acid

The main functionalities targeted in this assay were ethanol and acetic acid production in a cocoa pulp simulation medium (medium 1).

Medium Preparation

Medium 1: fructose (25 g/L), glucose (25 g/L), sucrose (75 g/L), citric acid (10 g/L), yeast extract (5 g/L) and soya peptone (5 g/L).

Yeast extract, neutralized soya peptone and sucrose were mixed together and the mix was sterilized at 120° C. for 20 min. After sterilization, pH was measured and adjusted if necessary to 6.5-7.

Fructose, glucose and citric acid were each autoclaved separately (120° C., 20 min) and aseptically added once temperature was around <50° C. to the sterilized mix. After mixing all medium components, pH was measured and adjusted to 3.5 if needed.

Assay I

Lab-scale cacao fermentation trials were carried out in 1 L baffled Erlenmeyer flasks loosely closed containing 150 ml cocoa pulp simulation medium (medium 1).

The Erlenmeyers were placed into an incubator at 25° C. The pH during fermentation was not regulated. The Erlenmeyers were agitated at 150-500 rpm.

Strains, previously propagated in the cocoa pulp simulation medium (medium 1), in 20 ml tubes at 25° C. during 20-30 h, were inoculated into the Erlenmeyer at a rate of 1-1.5% (vol/vol). Fermentation lasted 48 hours. Samples were taken at the beginning, during and at the end of fermentation.

Measurement of substrate consumption (glucose, fructose, sucrose) and metabolite production (acetic acid, ethanol) was carried out using a high performance liquid chromatography HPLC (pre-column: cation H cartridge 0 mm×4.6 mm and column: Aminex HPX-87H; Biorad) coupled to a wave length array spectrophotometer detector and a refractometer.

OD measurements at 600 nm were realized to follow yeast growth during fermentation. Samples taken at various times of the fermentation were diluted in Trypton salt (TS) and then were measured at 600 nm, in a Thermo Scientific Genesys 20™ spectrophotometer (with a spectral slit width specification of 8 nm and effective optical path of 10 mm); the OD (Optical Density) is calculated by multiplying the dilution factor of the sample by the value given by the spectrophotometer. Calculated OD is the multiplication of the dilution factor by the value given by the spectrophotometer.

Yeast Selection

Yeast were classified and selected according to the following criteria:

1. yeast producing ethanol,

2. yeast producing acetic acid, and

3. among yeast producing ethanol and acetic acid, the ones producing the highest levels were first selected

### Example 2: Application of the Assay of Example 1 to Yeasts

Example 1 above was implemented using a range of 43 different yeast strains mentioned below as inoculum:
Dekkera anomala
Dekkera bruxellensis
Brettanomyces naardenensis
Hanseniaspora uvarum
Hanseniaspora guillermondii
Pichia kluyveri
Pichia membranifaciens
Pichia fermentans
Wickerhamomyces anomalus
Kluyveromyces lactis
Kluyveromyces marxianus
Kazachstania unispora
Debaryomyces hansenii
Candida famata
Candida utilis
Candida zeylanoides
Candida incommunis
Saccharomyces cerevisiae
Saccharomyces bayanus
Zygotorulaspora florentina
Candida insconspicua
Yarrowia lypolytica

Tables 1 and 2 list the strains which are able to produce high level of acetic acid and/or high level of ethanol, according to the definition of the invention.

Yeasts were first classified according to the level of acetic acid produced during assay I (Table 1). From the 43 tested yeasts, 18 yeasts produced a level of acetic acid equals to or above 0.7 g/L. Among these, 11 yeasts produce a level of acetic acid above 1 g/L and are from the following species: Saccharomyces bayanus, Pichia kluyveri, Pichia anomala, Dekkera bruxellensis, Kluyveromyces marxianus (Candida kefyr), Saccharomyces cerevisiae, Hanseniaspora guillermondii and Candida incommunis.

Yeasts were then classified according to the level of ethanol produced during assay I (Table 2). From the 43 tested yeasts, 9 yeasts produce a high level of ethanol equals to or above 30 g/L and are from the following species: Saccharomyces cerevisiae, Kluyveromyces marxianus (Candida kefyr), Saccharomyces florentinus, Saccharomyces bayanus and Candida utilis major.

10 strains selected from 7 species were selected and their level of acetic acid and ethanol production was determined using the conditions disclosed in assay I, except that the temperature was 30° C. or 35° C. Table 3 summarizes the data obtained.

As shown in Table 3, 2 subgroups can be characterized from the strains producing high acetic acid level:
(1) yeasts which produce high level of acetic acid and high level of ethanol, such as strains from species Saccharomyces bayanus, Saccharomyces cerevisiae and Kluyveromyces marxianus (Candida kefyr); and
(2) yeasts which produce high level of acetic acid and low level of ethanol, such as strains from species Dekkera bruxellensis, Hanseniaspora guillermondii, Pichia kluyveri and Pichia anomala.

### Example 3: Kinetics of Acetic Acid Production by Yeast Strains

Medium Preparation

Medium 1: fructose (25 g/L), glucose (25 g/L), sucrose (75 g/L), citric acid (10 g/L), yeast extract (5 g/L) and soya peptone (5 g/L).

Yeast extract, neutralized soya peptone and sucrose were mixed together and the mix was sterilized at 120° C. for 20 min. After sterilization, pH was measured and adjusted if necessary to 6.5-7.

Fructose, glucose and citric acid were each autoclaved separately (120° C., 20 min) and aseptically added once temperature was around <50° C. to the sterilized mix. After mixing all medium constituents, pH was measured and adjusted to 3.5 if needed.

Assay

Lab-scale cacao fermentation trials were carried out in 3 L-fermentors containing 2 L cocoa pulp simulation medium.

Fermentation temperature was regulated at 30° C. The pH was adjusted and further regulated at 3.5-4.0 by base addition.

Agitation was 250-500 rpm.

Air outlet was 0.1-1 vvm.

Strains, previously propagated in the cocoa pulp simulation medium were inoculated into the fermentor at a rate of 1-10% (vol/vol). Fermentations lasted 72 h. Samples were taken at the beginning, during and at the end of fermentation.

Measurement of substrate consumption (glucose, fructose, sucrose) and metabolite production (acetic acid, ethanol) was carried out using a high performance liquid chromatography HPLC (pre-column: cation H cartridge 0 mm×4.6 mm and column: Aminex HPX-87H; Biorad) coupled to an array detector and a refractometer. Measurement of substrates and metabolites can also be realized by an enzymatic method using a Gallery food.

OD measurements at 600 nm were realized to follow yeast growth during fermentation (as detailed in example 1).

Selection

Yeast were classified and selected according to the following criteria:
the highest production of acetic acid
the highest rate of acetic acid production

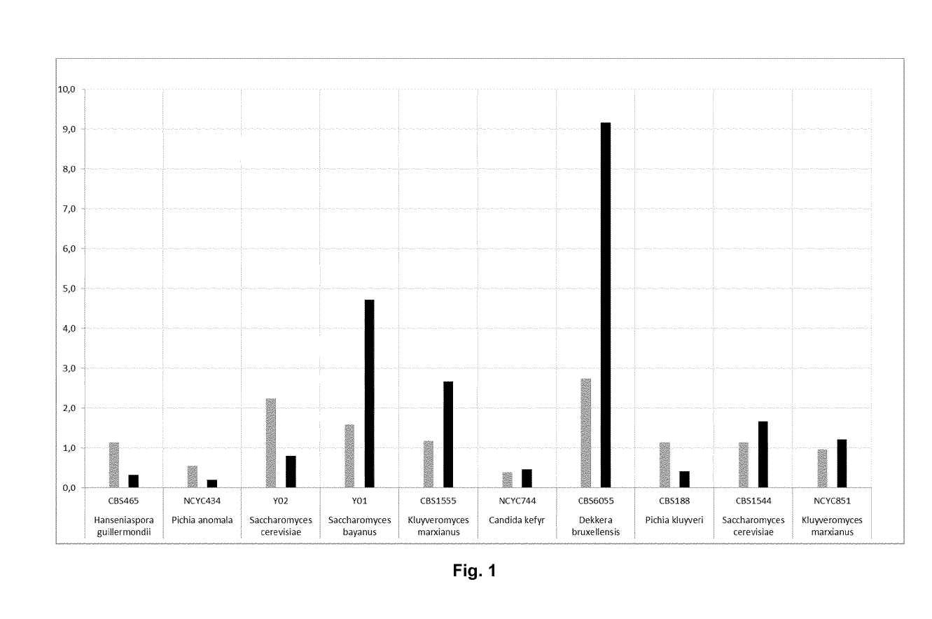

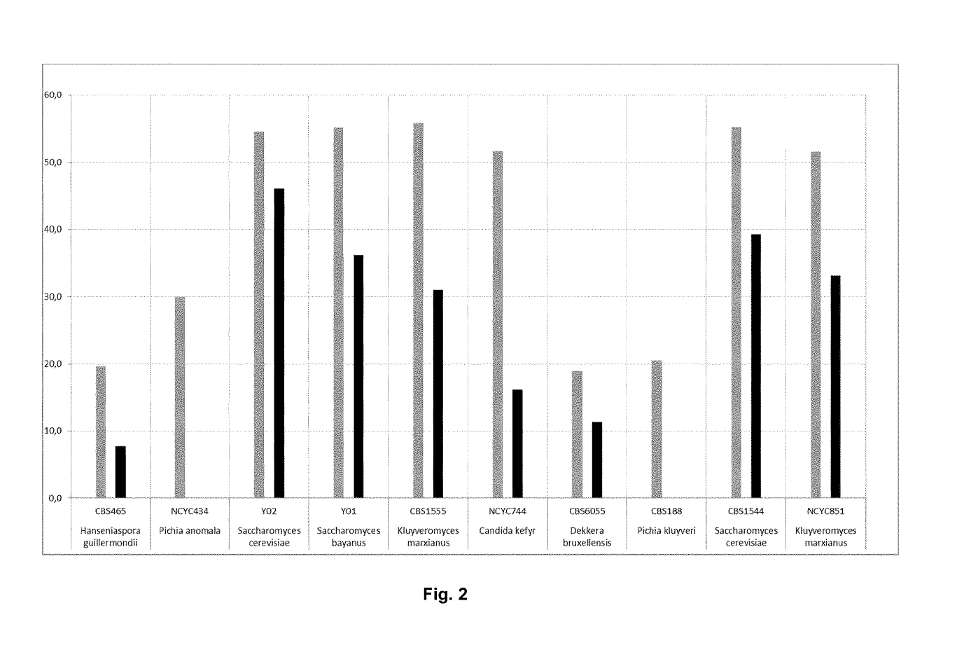

The same strains as the ones tested in Table 3 (example 2 above) were assayed for their kinetics in fermenters and data are summarized in Table 4 as well as in FIGS. 1 to 6. Comparisons of the acetic acid production and ethanol production (at 48 h) by the assay of example 1 (erlenmeyer) at 30° C. and the assay of example 3 (fermentor) for 10 strains are shown in FIGS. 1 and 2 respectively.

FIG. 1 shows that fermentor studies confirm the interest for Dekkera bruxellensis, Kluyveromyces marxianus and the Saccharomyces species (S. bayanus and S. cerevisiae), and to a lesser extent Hanseniaspora guillermondii, Pichia kluyveri and Pichia anomala.

FIG. 2 shows that fermentor studies confirm the interest for Saccharomyces and Kluyveromyces strain, and to a lesser extent Dekkera bruxellensis and Hanseniaspora guillermondii [similar profile between erlen and fermentor studies]. Both Pichia strains have been shown to be low ethanol-producers on fermentor experiments.

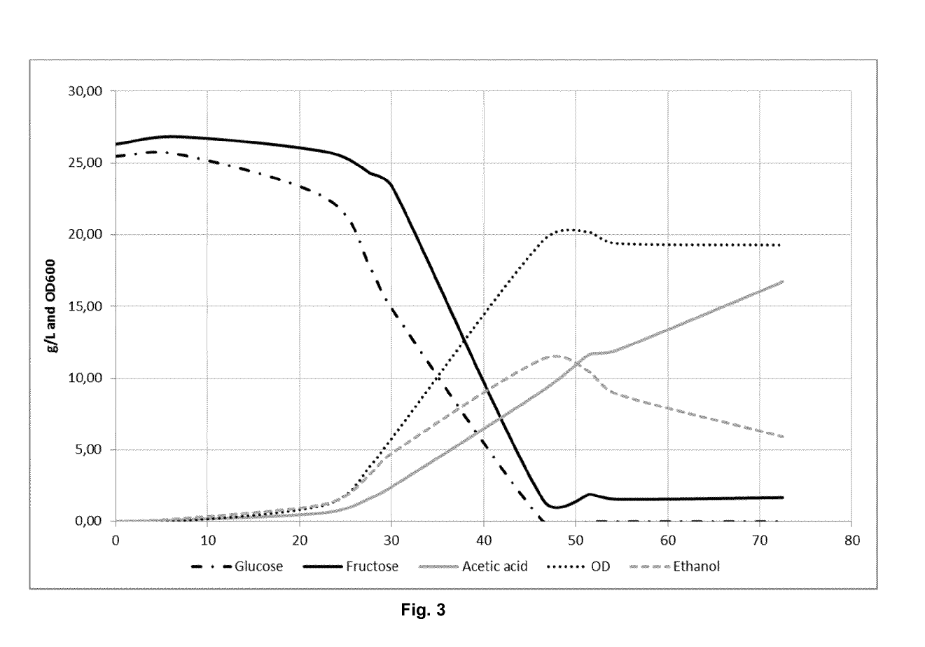

FIG. 3 shows the kinetics of acetic acid and ethanol production of Dekkera bruxellensis. Between 0 and 45 h, an increase in biomass (OD) and in acetic acid and ethanol production was observed. After 45 hours, a decrease in ethanol production and a stability of the biomass were observed, while the acetic acid production still increased (up to 16 g/L at 72 h). It is noteworthy that, at 45 h, all the fructose and glucose were consumed, suggesting that ethanol production was linked to the biomass production and sugar availability.

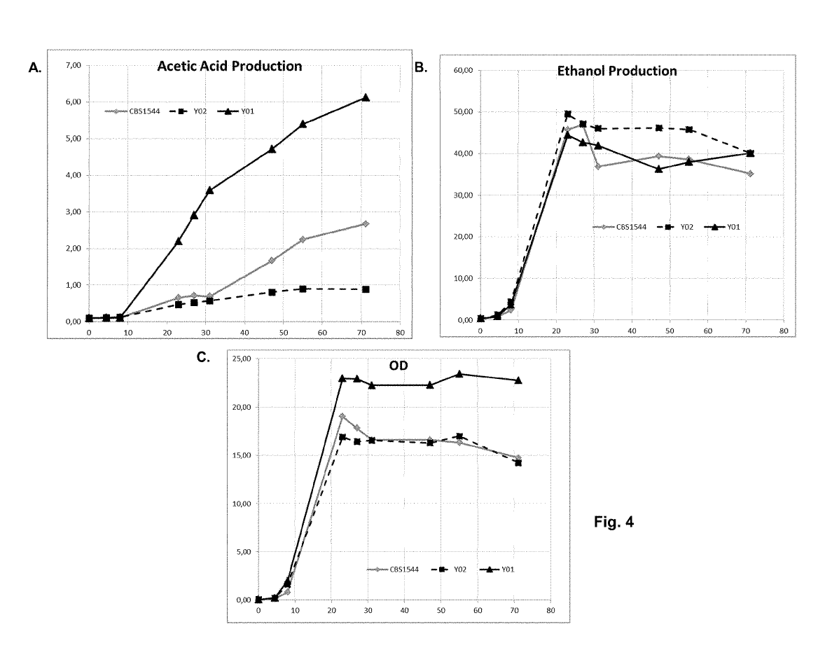

FIG. 4 shows the kinetics of acetic acid and ethanol production of 3 Saccharomyces strains—S. bayanus and S. cerevisiae. Between 0 and 25 h, an increase in biomass (OD) and in acetic acid and ethanol production was observed. After 25 h, the biomass stabilized. The ethanol production stayed around a high level (40 g/L for the 3 Saccharomyces strains), while the production of acetic acid kept on increasing to reach a concentration of 1 to 3 g/L (S. cerevisiae) and up to 6 g/L (S. bayanus) at 70 hours. All the glucose, fructose and sucrose were consumed at 25 h (data not shown). Interestingly, kinetics data show that S. bayanus is a better acid acetic producer than S. cerevisiae.

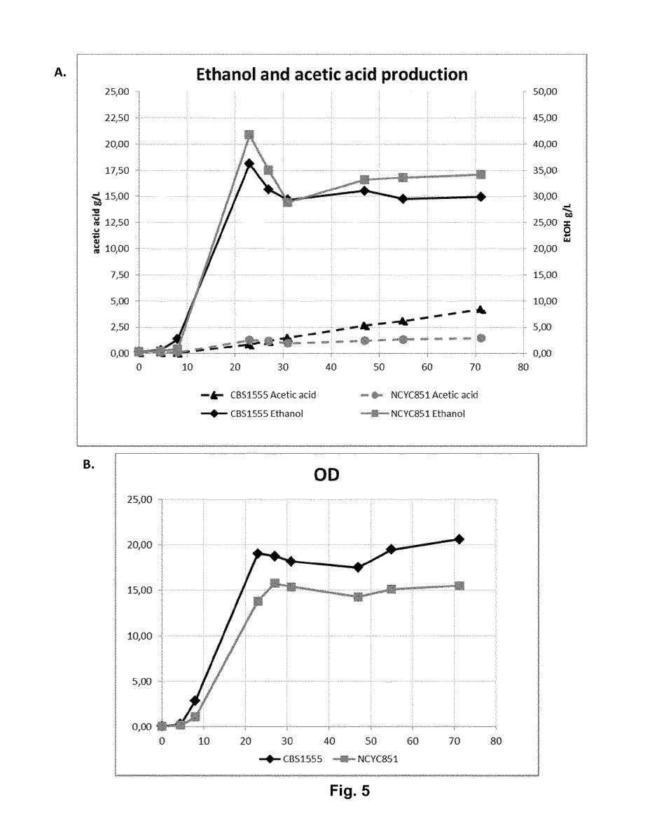

FIG. 5 shows the kinetics of acetic acid and ethanol production of 2 Kluyveromyces marxianus strains. Between 0 and 25 h, an increase in biomass (OD) and in acetic acid and ethanol production was observed. After 25 hours, a low decrease in ethanol production and a stability of the biomass were observed, while the acetic acid production still increased (up to 1 g/L for one strain and more than 4 g/l for the second strain). It is noteworthy that, at 25 h, all the fructose, glucose and sucrose were consumed (data not shown).

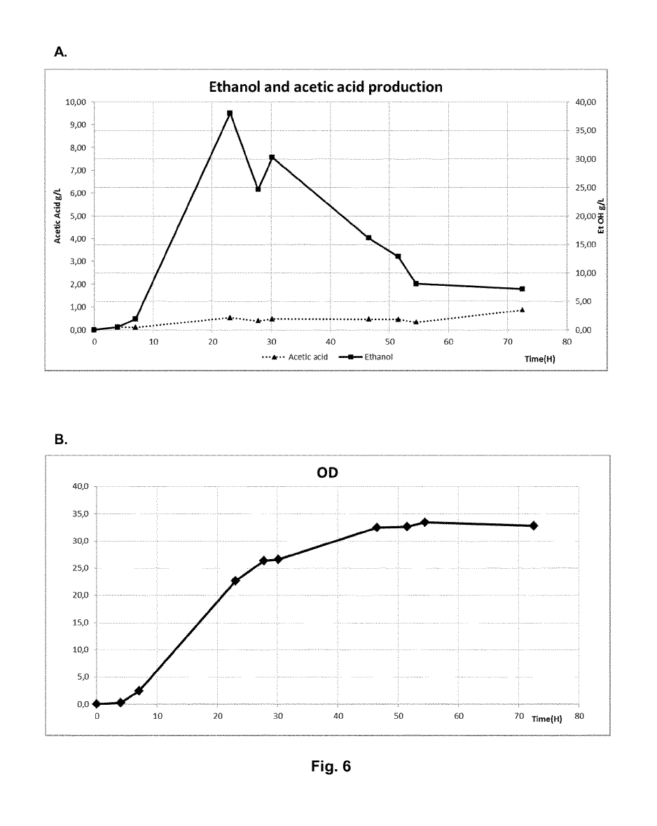

FIG. 6 shows the kinetics of acetic acid and ethanol production of Candida kefyr. Between 0 and 22 h, an increase in biomass (OD) was observed, consistent with the observations that at that time all the fructose, glucose and sucrose were consumed (data not shown). During the same period of time, an increase in ethanol production was observed, followed by a decrease after 22 h. The kinetics of acetic acid production is slow but regular and the level of acetic acid produced reaches 0.9 g/L at 72 h.

Other strains had not shown any significant production of acetic acid in the tested conditions (data not shown).

These assays have shown that the most interesting yeasts regarding kinetics of acetic acid production (and of ethanol production) in the tested conditions are Dekkera bruxellensis, Saccharomyces bayanus, Kluyveromyces marxianus (C. kefyr) and Saccharomyces cerevisiae.

### Example 4: Use of Selected Yeast Strains to Ferment Pasteurized Cocoa Pulp

During cocoa bean post-harvest fermentation, the main substrates for micro-organisms growth are provided by the pulp from the pods. The objective of this example was to check the ability of the previously selected yeasts to grow in cocoa pulp medium. In this order, pulp was pasteurized in order to destroy the indigenous micro-organisms of the pulp and to minimize their impact on the growth and activities of the selected added yeasts.

Pasteurized Cocoa Pulp Preparation

Frozen cocoa pulp was thawed in a water bath at 50° C. The pulp was then pasteurized by autoclave heating at a temperature of 105° C. during 5 minutes. The pasteurized pulp was then centrifuged at 4700 rpm during 10 min and the supernatant obtained was removed. The centrifugate was used as culture medium for yeast.

Assay

500 ml flasks were filled with the pasteurized pulp. Yeasts were inoculated at a concentration of 1E+05 cfu/ml of pulp. Incubation was performed, without stirring, in a water bath regulated at 32° C. After 48 h and 72 h of incubation, 10 ml were sampled in order to assess glucose, fructose, ethanol and acetic acid concentrations by using a high performance liquid chromatography HPLC (pre-column: cation H cartridge 0 mm×4.6 mm and column: Aminex HPX-87H; Biorad) coupled to an array detector and a refractometer. The yeast population in the flask was measured by plate count technique using PDA agar plates (potatoes Dextrose agar) for growth medium. The dishes were incubated during 48 h at 25° C.

Table 5 summarizes the acetic acid and ethanol production, glucose and fructose consumption and total yeast population of 5 different yeasts fermented on pasteurized cocoa pulp, at 48 h and 72 h (H. guillermondi CBS465 was tested in two separate experiments). These data represent the behaviour of the added yeast, as the indigenous flora was destroyed by pasteurizing the cocoa pulp. These data show that:
all added yeasts produced high level of acetic acid at both 48 h and 72 h
all added yeast produced high level of ethanol at both 48 h and 72 h
both fructose and glucose were totally consumed at 72 h; and
all added yeasts grew from an initial 10⁵ cfu/ml to a final 10⁷ up to 5.9 10⁸ cfu/ml of pulp.

Altogether, these data confirm that the medium and methods detailed in examples 1 and 3 herein, in particular the assay I described herein, are satisfying models mimicking the behaviour of yeasts on cocoa pulp substrate.

### Example 5: Use of Selected Yeast Strains to Ferment Raw Cocoa Pulp

The objective of this assay was to check that the addition of the previously selected yeasts to raw cocoa pulp (containing indigenous micro-organisms) leads to an early increase of both acetic acid and ethanol, as compared to a control.

Raw Cocoa Pulp Preparation

Frozen cocoa pulp was thawed in a water bath at 50° C.

Assay

500 ml flasks were filled with the raw pulp. Yeasts were inoculated at a concentration of 1E+05 cfu/ml of pulp. For each experiment, an assay without yeast addition was done as a negative control. Incubation was performed, without stirring, in a water bath regulated at 32° C. After 48 h and 72 h of incubation, 10 ml were sampled in order to assess glucose, fructose, ethanol and acetic acid concentrations as detailed in example 4. The yeast population in the flask is measured as detailed in example 4.

Table 6 summarizes the acetic acid and ethanol production, and the glucose and fructose consumption of 4 different yeasts fermented on raw (non-pasteurized) cocoa pulp, at 48 h and 72 h (H. guillermondi CBS465 was tested in two separate experiments), with their respective control (no yeast addition). Table 6 also summarizes the total yeast population (cfu) at t=0 (indigenous yeasts) and at 48 h and 72 h. These data represent the behaviour of both the added yeast and the indigenous flora present in the cocoa pulp. The population represents the total number of yeasts, i.e., the growth of the added yeast and the indigenous yeasts.

These data show that:
at both 48 h and 72 h, a statistically significant higher level of ethanol was produced when a yeast according to the invention is added, as compared to the control;
at both 48 h and 72 h, a statistically significant higher level of acetic acid was produced when D. bruxellensis yeast is added, as compared to the control;
the level of acetic acid produced at both 48 h and 72 h was increased by the addition of K. marxianus or C. kefyr as compared to the control;
fructose and glucose were highly consumed at 72 h when a yeast according to the invention is added, as compared to the control.

Altogether, these data confirm the interest for the use of D. bruxellensis in a cocoa fermentation process, and to a lesser extent for the use of K. marxianus and C. kefyr.

### Example 6: Use of Selected Yeast Strains to Ferment Cocoa Beans

This example illustrates a method for fermenting cocoa beans using yeasts strain(s) according to the invention as compared to a natural spontaneous fermentation.

Cocoa pods (for example Ivory Coast, Forastero) are opened and the cocoa beans surrounded by pulp are transferred into a fermentation box.

Different yeast inoculums are prepared. Yeast under powder form is rehydrated in a saline solution.

Each yeast inoculum is then added to a separate fermentation box, at a rate of 10⁵ to 10⁸ CFU/g plant material. Beans are mixed thoroughly to ensure homogeneity. A fermentation box non-inoculated is used as a control.

After 2 and 4 days, the beans are turned to allow for homogeneous fermentation and for air to enter.

At regular times, for example every day, beans samples are taken to check the level of liquefaction of the pulp.

The fermentation duration is determined as when 80%, 90% of the beans are free of pulp (meaning the pulp is completely liquefied).

At the end of fermentation, the beans are collected and then dried (e. g. by sun drying) until their moisture content is <9%.

The assessment of bean overall quality is established by means of a cut-test. Appearance and more precisely color of the beans indicate if fermentation went well.

A normal spontaneous fermentation lasts 6 to 7 days; however it is expected that the duration of cocoa fermentation to which a yeast starter has been added according to the present invention will be reduced by 1 or 2 days. The results of this example are expected to illustrate that the fermentation of cocoa beans with the use of a yeast according to the present invention is much faster than a spontaneous fermentation.

TABLE 1
Acetic acid production, OD (600 nm) and ratio acetic acid/OD of strains tested by assay I

| | | | | Acetic Acid | Acetic acid/OD |
|---|---|---|---|---|---|
| Species | other name | Strain | OD | (g/L) | (mg/L/OD) |
| Saccharomyces bayanus | / | Y01 | 17.0 | 2.7 | 160.5 |
| Pichia kluyveri | / | CBS 188 | 4.8 | 2.6 | 543.7 |
| Pichia anomala | Wickerhamomyces anomalus | NCYC434 | 13.3 | 2.0 | 151.4 |
| Dekkera bruxellensis | Brettanomyces bruxellensis | CBS 6055 | 4.6 | 1.9 | 409.1 |
| Kluyveromyces marxianus | Candida kefyr | CBS1555 | 12.4 | 1.4 | 116.3 |
| Candida kefyr | Kluyveromyces marxianus | NCYC744 | 11.1 | 1.3 | 120.9 |
| Saccharomyces cerevisiae | / | CBS 1544 | 11.1 | 1.2 | 106.6 |
| Kluyveromyces marxianus | Candida kefyr | NCYC851 | 9.5 | 1.2 | 122.5 |
| Saccharomyces cerevisiae | / | Y02 | 13.3 | 1.1 | 84.3 |
| Hanseniaspora guillermondii | Kloeckera apis | CBS 465 | 5.2 | 1.1 | 206.9 |
| Candida incommunis | / | CBS 5604 | 10.0 | 1.1 | 107.2 |
| Dekkera anomala | Brettanomyces anomalus, | CBS 1947 | 0.8 | 0.8 | 1049.8 |
| | Brettanomyces claussenii | | | | |
| Candida utilis major | Lindnera jadinii | Y03 | 13.1 | 0.8 | 59.2 |
| Dekkera anomala | Brettanomyces anomalus, | CBS 4460 | 1.8 | 0.8 | 424.2 |
| | Brettanomyces claussenii | | | | |
| Hanseniaspora guillermondii | Kloeckera apis | CBS 1972 | 10.7 | 0.7 | 65.0 |
| Pichia fermentans | Candida lambica | CBS 187 | 7.6 | 0.7 | 90.2 |
| Hanseniaspora uvarum | Kloeckera apiculata | CBS 314 | 6.4 | 0.7 | 106.2 |
| Pichia fermentans | Candida lambica | Y04 | 9.6 | 0.7 | 68.5 |
| Saccharomyces florenlinus | Zygotorulaspora florentina | Y05 | 14.8 | 0.4 | 29.8 |
| Saccharomyces cerevisiae | / | Y06 | 12.5 | 0.2 | 19.9 |

TABLE 2
Ethanol production, OD (600 nm) and ratio ethanol/OD of strains tested by assay I

| | | | | Ethanol | Ethanol/OD |
|---|---|---|---|---|---|
| Species | other names | Strain | OD | (g/L) | (g/L/OD) |
| Saccharomyces cerevisiae | / | Y02 | 13.3 | 55.6 | 4.2 |
| Candida kefyr | Kluyveromyces marxianus | NCYC 744 | 11.1 | 54.9 | 5.0 |
| Saccharomyces florentinus | Zygotorulaspora florentina | Y05 | 14.8 | 53.8 | 3.6 |
| Saccharomyces bayanus | / | Y01 | 17.0 | 51.0 | 3.0 |
| Saccharomyces cerevisiae | / | CBS 1544 | 11.1 | 50.5 | 4.5 |
| Saccharomyces cerevisiae | / | Y06 | 12.5 | 49.0 | 3.9 |
| Kluyveromyces marxianus | Candida kefyr | CBS1555 | 12.4 | 48.2 | 3.9 |
| Kluyveromyces marxianus | Candida kefyr | NCYC851 | 9.5 | 46.0 | 4.8 |
| Candida utilis major | Lindnera jadinii | Y03 | 13.1 | 39.0 | 3.0 |
| Pichia fermentans | Candida lambica | CBS 187 | 7.6 | 21.5 | 2.8 |
| Pichia fermentans | Candida lambica | Y04 | 9.6 | 20.9 | 2.2 |
| Hanseniaspora guillermondii | Kloeckera apis | CBS 1972 | 10.7 | 18.8 | 1.8 |
| Hanseniaspora guillermondii | Kloeckera apis | CBS 465 | 5.2 | 18.3 | 3.5 |
| Candida incommunis | / | CBS 5604 | 10.0 | 17.1 | 1.7 |
| Pichia kluyveri | / | CBS 188 | 4.8 | 16.7 | 3.5 |
| Hanseniaspora uvarum | Kloeckera apiculata | CBS 314 | 6.4 | 15.0 | 2.3 |
| Pichia anomala | Wickerhamomyces anomalus | NCYC434 | 13.3 | 10.1 | 0.8 |
| Dekkera bruxellensis | Brettanomyces bruxellensis | CBS 6055 | 4.6 | 4.0 | 0.9 |
| Dekkera anomala | Brettanomyces anomalus, | CBS 4460 | 1.8 | 1.8 | 1.0 |
| | Brettanomyces claussenii | | | | |
| Dekkera anomala | Brettanomyces anomalus, | CBS 1947 | 0.8 | 0.8 | 1.0 |
| | Brettanomyces claussenii | | | | |

TABLE 3
Acetic acid and ethanol production, and OD (600 nm) of strains tested by a
assay similar to assay I (30 and 35° C.)

| | | 30° C., 48 h | | | 35° C., 48 h | | |
|---|---|---|---|---|---|---|---|
| | | acetic acid | ethanol | | acetic acid | ethanol | |
| Species | strain | (g/L) | (g/L) | OD | (g/L) | (g/L) | OD |
| D. bruxellensis | CBS 6055 | 2.75 | 17.32 | 11.7 | 2.3 | 20.5 | 12.4 |
| H. guillermondii | CBS 465 | 1.10 | 18.65 | 5.1 | 1.02 | 18.04 | 3.5 |
| P. kluyveri | CBS 188 | 1.06 | 18.17 | 5.2 | 0.17 | <0.02 | 0.7 |
| P. anomala | NCYC434 | 0.60 | 25.68 | 8.4 | 0.10 | 6.45 | 1 |
| S. cerevisiae | CBS 1544 | 0.90 | 53.87 | 10.8 | 0.53 | 49.95 | 6.5 |
| S. cerevisiae | Y02 | 1.45 | 53.19 | 12.20 | 0.42 | 51.23 | 10.4 |
| S. bayanus | Y01 | 2.94 | 50.73 | 16.3 | 1.57 | 54.02 | 12.3 |
| K. marxianus | NCYC851 | 0.99 | 45.41 | 11.6 | 1.05 | 45.22 | 10.5 |
| K. marxianus | CBS1555 | 1.13 | 55.05 | 10.5 | 1.13 | 50.70 | 11.2 |
| C. kefyr | NCYC 744 | 0.79 | 50.12 | 11.3 | 1.02 | 47.56 | 12 |

TABLE 4
Acetic acid and ethanol production, and OD (600 nm) of strains tested in
fermentor (30° C.)

| | | 48 hours | | | 72 hours | | |
|---|---|---|---|---|---|---|---|
| | | | Ethanol | Acetic Acid | | Ethanol | Acetic Acid |
| Species | strain | OD | (g/L) | (g/L) | OD | (g/L) | (g/L) |
| Dekkera bruxellensis | CBS 6055 | 19.6 | 11.4 | 9.2 | 19.3 | 5.9 | 16.7 |
| Hanseniaspora guillermondii | CBS 465 | 11.8 | 7.8 | 0.3 | 10.2 | 2.9 | 0.4 |
| Pichia kluyveri | CBS 188 | 18.5 | <0.02 | 0.4 | 18.0 | <0.02 | 0.3 |
| Pichia anomala | NCYC434 | 11.5 | <0.02 | 0.2 | 28.0 | 0.3 | 0.1 |
| Saccharomyces cerevisiae | CBS 1544 | 16.6 | 39.3 | 1.7 | 14.7 | 35.1 | 2.7 |
| Saccharomyces cerevisiae | Y02 | 16.3 | 46.1 | 0.8 | 14.2 | 40.0 | 0.9 |
| Saccharomyces bayanus | Y01 | 22.3 | 36.2 | 4.7 | 22.8 | 40.0 | 6.1 |
| Kluyveromyces marxianus | NCYC851 | 17.5 | 31.0 | 2.7 | 20.6 | 29.9 | 4.2 |
| Kluyveromyces marxianus | CBS1555 | 14.3 | 33.1 | 1.2 | 15.5 | 34.1 | 1.5 |
| Candida kefyr | NCYC 744 | 32.4 | 16.1 | 0.5 | 32.8 | 7.2 | 0.9 |

TABLE 5
Acetic acid and ethanol production, glucose and fructose consumption and total
population (pop) of yeasts fermented on pasteurized cocoa pulp (at 48 h and 72 h)

| | | 48 hours | | | | | 72 hours | | | | |
|---|---|---|---|---|---|---|---|---|---|---|---|
| | | Glucose | Fructose | Ethanol | Acetic | | Glucose | Fructose | Ethanol | Acetic | |
| Species | Strain | (g/L) | (g/L) | (g/L) | Acid (g/L) | Pop cfu | (g/L) | (g/L) | (g/L) | Acid (g/L) | Pop cfu |
| H. guillermondii | CBS 465 | 0.38 | 0.56 | 45.08 | 1.16 | 3.3E+07 | 0.42 | 0.57 | 44.28 | 1.08 | 3.7E+07 |
| H. guillermondii | CBS 465 | 12.64 | 15.12 | 41.17 | 1.20 | 6.3E+07 | 0.79 | 0.76 | 49.74 | 1.35 | 1.4E+07 |
| D. bruxellensis | CBS 6055 | 16.64 | 35.58 | 18.58 | 0.86 | 5.0E+08 | 0.41 | 1.41 | 42.81 | 0.99 | 5.9E+08 |
| K. marxianus | CBS1555 | 6.07 | 14.92 | 33.01 | 1.28 | 1.1E+08 | 0.31 | 0.34 | 41.52 | 1.44 | 1.0E+08 |
| K. marxianus | NCYC851 | 11.91 | 21.28 | 28.65 | 1.14 | 9.0E+07 | 0.59 | 4.50 | 42.01 | 1.47 | 1.4E+08 |
| C. kefyr | NCYC 744 | 4.24 | 17.13 | 33.01 | 1.32 | 2.1E+08 | 0.31 | 0.35 | 41.72 | 1.57 | 2.1E+08 |

TABLE 6

| | Acetic acid and ethanol production, glucose and fructose consumption and total | | | | | | | | | | | | |
|---|---|---|---|---|---|---|---|---|---|---|---|---|---|
| | population (pop) of yeasts fermented on raw cocoa pulp (at 48 h and 72 h) | | | | | | | | | | | | |
| | | | | 48 hours | | | | | 72 hours | | | | |
| | | | | | | | Acetic | | | | | Acetic | |
| | Added | | Pop cfu | Glucose | Fructose | Ethanol | Acid | | Glucose | Fructose | Ethanol | Acid | |
| | yeast | Strain | at t = 0 | (g/L) | (g/L) | (g/L) | (g/L) | Pop cfu | (g/L) | (g/L) | (g/L) | (g/L) | Pop cfu |
| 1 | None (control) | / | 9.00E+02 | 48.97 | 57.88 | 0.19 | 1.00 | 5.50E+05 | 46.82 | 55.46 | 1.95 | 1.90 | 4.20E+06 |
| | D. bruxellensis | CBS 6055 | | 31.17 | 51.21 | 9.33 | 4.74 | 4.00E+05 | 11.08 | 31.04 | 28.55 | 5.40 | 1.60E+08 |
| 2 | None (control) | / | <1.00E+02 | 43.62 | 45.48 | 0.81 | 1.20 | 1.12E+06 | 35.29 | 38.06 | 8.60 | 2.71 | 3.10E+06 |
| | D. bruxellensis | CBS 6055 | | 22.42 | 40.28 | 12.38 | 3.86 | 1.10E+06 | 5.52 | 2.59 | 36.46 | 4.39 | 1.31E+08 |
| 3 | None (control) | / | 5.00E+02 | 45.46 | 55.25 | 0.00 | 0.94 | 4.4E+05 | 41.67 | 53.11 | 2.97 | 1.30 | 5.3E+06 |
| | K. marxianus | CBS1555 | | 1.76 | 5.80 | 43.38 | 1.35 | 9.9E+07 | 0.66 | 0.17 | 47.72 | 1.49 | 4.5E+07 |
| | K. marxianus | NCYC851 | | 5.74 | 16.30 | 40.19 | 1.26 | 1.3E+08 | 0.64 | 0.20 | 49.47 | 1.53 | 9.8E+07 |
| | C. kefyr | NCYC744 | | 0.77 | 4.17 | 46.70 | 1.52 | 1.5E+08 | 0.68 | 0.17 | 47.41 | 1.59 | 1.2E+08 |

1. A method for the fermentation of cocoa beans comprising the steps of:
a) adding at least one yeast to a plant material comprising:
(i) beans derived from fruit pods of the species Theobroma cacao; and
(ii) pulp; and
b) fermenting the plant material to obtain fermented cocoa beans, the fermentation environment containing at least one other microorganism selected from the group consisting of yeast, lactic acid bacteria and acetic acid bacteria;
wherein the added yeast is able to produce ethanol and acetic acid by fermentation of a carbohydrate.

2. A method according to claim 1, wherein said added yeast is able:
to produce acetic acid during assay I in a concentration of at least 0.7 g/L, and/or
to produce acetic acid during assay I in a ratio of the concentration of acetic acid produced by said yeast over the final OD of at least 50 mg/L/OD.

3. The method according to claim 1, wherein no acetic acid bacterium is added during the fermentation step.

4. The method according to claim 1, wherein more than 60% of the acetic acid produced during the fermentation of cocoa beans is carried out by said at least one added yeast.

5. The method according to claim 1, wherein the ratio (log/log) total yeast/total acetic acid bacteria present in the fermentation environment is more than 0.8 during the 3 first days of fermentation.

6. The method according to claim 1, wherein said at least one yeast is from a genus selected from the group consisting of Dekkera, Brettanomyces, Hanseniaspora, Kloeckera, Pichia, Candida, Saccharomyces, Kluyveromyces, Debaryomyces, Kazachstania, Wickerhamomyces, Lindnera and Zygotorulaspora, or any mixture thereof.

7. The method according to claim 1, wherein said at least one yeast is from a species selected from the group consisting of Dekkera bruxellensis (anamorph Brettanomyces bruxellensis), Brettanomyces custersianus, Brettanomyces nanus, Brettanomyces naardenensis, Hanseniaspora uvarum (anamorph Kloeckera apiculata), Hanseniaspora guillermondii (anamorph Kloeckera apis), Hanseniaspora osmophila (anamorph Kloeckera cortisis), Pichia fermentans (anamorph Candida lambica), Pichia membranifaciens (anamorph Candida valida), Wickerhamomyces anomalus, Pichia kluyveri, Kluyveromyces lactis, Debaryomyces hansenii (anamorph Candida famata), Lindnera jadinii (anamorph Candida utilis), Kazachstania unispora, Zygotorulaspora florentina, and Saccharomyces bayanus or a mixture of any thereof.

8. The method according to claim 1, wherein said at least one yeast has a pectinolytic activity.

9. The method according to claim 1, wherein:
at least one other yeast is added in step (a), and
said other yeast has a pectinolytic activity.

10. The method according to claim 1, wherein at least one enzyme having a pectinolytic activity is added in step (a).

11. A method of producing a cocoa-based product, wherein the method comprises:
(a) providing fermented cocoa beans obtained or obtainable by a method according to claim 1, and
(b) carrying out one or more further processing steps on said fermented cocoa beans to produce the cocoa-based product.

12. The method according to claim 11 wherein the one more more further processing steps of (b) comprise(s) drying said fermented cocoa beans.

13. The method according to claim 11, wherein the method comprises a further step of thermally treating the dried beans.

14. The method according to claim 13, wherein the method comprises further steps of:
removing shells from the roasted beans to produce cocoa nibs, and
grinding the nibs to produce a cocoa liquor.

15. (canceled)

16. A mixture of yeasts, wherein the mixture comprises at least 2 yeasts of different species selected independently from the group consisting of Saccharomyces bayanus, Saccharomyces cerevisiae, Kluyveromyces marxianus (Candida kefyr), Dekkera bruxellensis, Hanseniaspora guillermondii, Pichia kluyveri and Pichia anomala.

17. A mixture of yeasts according to claim 16, wherein:
one yeast is selected from the group consisting of Saccharomyces bayanus and Dekkera bruxellensis; and
one yeast is selected from the group consisting of Saccharomyces bayanus, Dekkera bruxellensis, Kluyveromyces marxianus (Candida kefyr), Saccharomyces cerevisiae, Hanseniaspora guillermondii, Pichia kluyveri and Pichia anomala.

18. A method according to claim 1, wherein said added yeast is able:
to produce ethanol during assay I in a concentration of at least 30 g/L, and/or
to produce ethanol during assay I in a ratio of the concentration of ethanol produced by said yeast over the final OD of at least 3 g/L/OD.

19. The method according to claim 1, wherein the ratio (log/log) total yeast/total acetic acid bacteria present in the fermentation environment is more than 0.8 during the whole fermentation step.

20. The method according to claim 1, wherein said at least one yeast is a species selected from the group consisting of Dekkera bruxellensis and Saccharomyces bayanus.

21. The method according to claim 1, wherein said at least one yeast is selected from the group consisting of a mixture of yeasts comprising Dekkera bruxellensis and a mixture of yeasts comprising Saccharomyces bayanus.

22. The method according to claim 7, wherein any yeast species from Pichia kluyveri is other than the strains Pichia kluyveri PK-KR1 (V06/022711) and Pichia kluyveri PKKR2 (V06/022712).
